(19) United States
(12) Patent Application Publication
(10) Pub. No.: US 2025/0320542 A1
(43) Pub. Date: Oct. 16, 2025

(54) SPATIALLY BARCODED SURFACES

(71) Applicant: Cellanome, Inc., Foster City, CA (US)

(72) Inventor: Stephen C. MACEVICZ, Bozeman, MT (US)

(21) Appl. No.: 19/252,879

(22) Filed: Jun. 27, 2025

Related U.S. Application Data
(63) Continuation of application No. PCT/US2023/086095, filed on Dec. 27, 2023.
(60) Provisional application No. 63/436,428, filed on Dec. 30, 2022.
(60) Provisional application No. 63/457,067, filed on Apr. 4, 2023.

(51) Int. Cl. C12Q 1/6806

(52) U.S. Cl. CPC C12Q 1/6806

(57) ABSTRACT

The systems and methods described herein are directed to methods of making spatially barcoded surfaces by a spatially restricted release of unique oligonucleotide barcodes from beads.

### CROSS-REFERENCE

This application is a continuation of International Application No. PCT/US2023/086095, filed Dec. 27, 2023, which claims the benefit of U.S. Provisional Application No. 63/436,428, filed Dec. 30, 2022, and U.S. Provisional Application No. 63/457,067, filed Apr. 4, 2023, which are incorporated herein by reference in entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

The use of spatial barcodes to study spatial differences in cellular processes, such as gene expression, has expanded tremendously in recent years, e.g. Williams et al, Genome Medicine, 14:68 (2022); Li et al, Biophys. Rep., 8 (3): 119-135 (2022); Tian et al, Nature Biotechnology, (https://doi.org/10.1038/s41587-022-01448-2) (2022). Many approaches have been developed for spatially barcoding surfaces in such studies which vary widely in spatial resolution, convenience of production and use, and the nature of spatial information recovered, e.g. Stahl et al, Science, 353 (6294): 78-82 (2016); Rodriques et al, Science, 363 (6434): 1463-1467 (2019); Lui et al, Cell, 183:1665-1681 (2020); Chen et al, Cell, 185:1777-1792 (2022); Fu et al, Cell, 185:4621-4633 (2022); and the like. Most approaches provide surfaces with attached oligonucleotide barcodes that encode spatial information and that are capable of interacting with molecules of interest which, in turn, result in products which may then analyzed (for example, by sequencing) to identify the molecule and its location on the surface or its association with other molecules having the same barcode. Unfortunately, currently available approaches all suffer from one or more drawbacks because of technique complexity, expense, resolution and/or convenience, e.g. Kleino et al, Computational and Structural Biotechnology Journal, 20:4870-4884 (2022).

In view of the above, numerous biological and medical applications of spatial barcoding would be advanced with the availability of a flexible and convenient method for making spatially barcoded surfaces.

### SUMMARY

The methods, compositions and kits described herein are for making and using spatially barcoded surfaces. In some embodiments, methods comprise: (a) providing a surface comprising capture oligonucleotides attached thereto and a plurality of closely spaced beads disposed thereon, wherein each bead comprises releasably attached barcode oligonucleotides each comprising a barcode sequence; (b) releasing the barcode oligonucleotides so that the barcode oligonucleotides are captured by capture oligonucleotides; and (c) surface amplifying the captured barcode oligonucleotides to form clonal populations of captured barcode oligonucleotides on the surface. In some embodiments, beads are spaced from one another. In some embodiments, beads are closely spaced (or closely packed), as described below. In some embodiments, beads comprising barcode oligonucleotides are mixed with spacer beads. In some embodiments, the method further comprising adding to said surface a diffusion inhibitor that occupies interstitial spaces between said beads. In some embodiments, on each of the beads a portion of the barcode oligonucleotides have a 5′ phosphate. In some embodiments, the method further comprises providing a splint oligonucleotide configured to form a duplex with a 5′-end and a 3′-end of the barcode oligonucleotide so that the 5′-end and the 3′-end are linked in the presence of a ligase to form a tandem barcode oligonucleotide whenever the 5′-end of the barcode oligonucleotide has a 5′ phosphate.

Further described herein is a composition comprising a surface partitioned into a plurality of sub-regions (i.e., the surface is covered by a tiling or quasi-tiling) wherein each different sub-region (or tile) of the surface has attached a different primary barcode oligonucleotide and each sub-region (or tile) has attached at least one tandem barcode oligonucleotide from at least one adjacent sub-region (or tile). In some embodiments, each sub-region (or tile) has attached at least one tandem barcode oligonucleotide from every adjacent sub-region (or tile). In some embodiments, a surface is partitioned or covered by a tiling of regular polygons. In some embodiments, such regular polygons are hexagons and each hexagonal sub-region has attached therein a plurality from 2 to 6 of secondary barcode oligonucleotides from adjacent hexagonal sub-regions, or tiles.

The present method provides inexpensive ways to construct spatially barcoded surfaces and, in some embodiments, provides a means for determining the relative positions of sub-regions, or tiles, containing barcode oligonucleotides.

### BRIEF DESCRIPTIONS OF THE DRAWINGS

FIG. 1 illustrates an embodiment using spherical beads for delivering barcode oligonucleotides to a surface and using hexagonal tiles for analyzing barcode positions.

FIG. 2A illustrates an example of a structure of a barcode oligonucleotide which can be released from a bead as a “seed” oligonucleotide for the formation of clusters on a surface for transcriptome analysis.

FIG. 2B illustrates how the example barcode oligonucleotide of FIG. 2A may be used to generate barcode clusters on a surface.

FIG. 2C illustrates an embodiment in which some barcode oligonucleotides may be ligated so that when released with splint oligonucleotides in the presence of a ligase activity tandem barcode oligonucleotides are formed which comprise barcode sequences from adjacent beads.

FIG. 2D illustrates three different barcode-containing oligonucleotide strands that may be attached to a surface in border regions between tiles.

FIG. 2E illustrates a protocol for blocking a cleavage site in an oligonucleotide strand containing tandem barcodes.

FIG. 3A illustrates a surface divided into hexagonal sub-regions (“tiles”) each corresponding to the presence of a bead comprising oligonucleotides each comprising the same unique barcode sequence. Table 1 gives barcode compositions for tile “b4” and for its surrounding tiles assuming that 3 percent of barcode oligonucleotides released by a given bead “leak” into each adjacent tile.

FIG. 3B is a flow chart of an algorithm for determining the relative positions of barcode tiles on a surface using information from tandem barcode oligonucleotides.

FIGS. 3C-3D illustrate in terms of tiles the operation of the algorithm of FIG. 3B.

FIG. 4 illustrates an application of spatially barcoded surfaces.

FIG. 5 illustrates an example of a flow cell with a plurality of channels, each comprising a surface on which bead may be disposed in closely packed arrays.

### DETAILED DESCRIPTION

The practice of the present systems and methods described herein may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation and use of synthetic nucleotides, polynucleotides, molecular conjugation, surface chemistries, and the like. Specific illustrations of suitable techniques can be had by reference to the example herein below. However, other equivalent conventional procedures can also be used. Such conventional techniques, materials and descriptions can be found in standard laboratory manuals including, but not limited to, Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Moore et al, Building Scientific Apparatus, Third Edition (Perseus Books, Cambridge, MA); Hermanson, Bioconjugate Techniques, 3rd Edition (Academic Press, 2013); and like references.

Described herein are methods, compositions and systems for producing and using spatially barcoded surfaces using beads or particles to transfer barcodes to limited sub-regions of such surfaces. As used herein, the term “spatially barcoded surface” means a surface having attached molecular indicators from which surface position may be determined or indicated. In some embodiments, such molecular indicators comprise surface location information encoded in a polymer sequence, such as, an oligonucleotide sequence. In some embodiments, a plurality of sub-regions, or tiles, of a surface may be formed in which the same unique molecular indicator is present or predominant, that is, such that different sub-regions or tiles each have substantially different molecular indicators. In other embodiments, a plurality of sub-regions, or tiles, of a surface may be formed in which there is a primary, or predominant, molecular indicator present as well as a number of secondary molecular indicators present by diffusion from adjacent tiles. In some embodiments, information from such secondary molecular indicators is used to determine or to calculate the relative positions of the primary molecular indicators, and/or their corresponding tiles, on a surface. As described more fully below, in some embodiments, such secondary molecular indicators take the form of tandem barcode oligonucleotides.

The terms “bead” and “particle” are used interchangeably to denote a discrete solid support typically used or manipulated in populations, but which may be separated from one another. In some embodiments, the term “beads” refers to a monodisperse population of approximately spherical particles typically having diameters with a coefficient of variation less than 25 percent, or in some embodiments, less than 10 percent, or in some embodiments less than 5 percent, or in some embodiments, less than 2 percent. In some embodiments, such beads may have diameters in the range of from 0.01 μm to 200 μm. In some embodiments, such beads may have diameters in the range of from 1 μm to 100 μm. In other embodiments, such beads may have diameters in the range of from 10 μm to 100 μm. The composition of beads used may vary widely. In some embodiments, beads may be non-porous, so that synthesis or attachment of barcode oligonucleotides takes place substantially only on bead surfaces, or in other embodiments, beads may be porous, so that synthesis or attachment of barcode oligonucleotides takes place not only on beads surfaces, but also throughout the interiors of the beads. In some embodiments, porous beads comprise hydrogel beads. In some embodiments, hydrogel beads are degradable hydrogel beads, e.g. as disclosed in U.S. patent publications, US2014/0378322; 11180752; Kharkar et al, Chem. Soc. Rev., 42:7335 (2013); which references are incorporated by reference. In various embodiments, hydrogel beads may be chemically degradable beads, enzymatically degradable beads, photodegradable beads, and/or thermally degradable beads. In some embodiments, beads may comprise chemically reducible cross-linkers such, as for example, chemically reducible cross-linkers that comprise disulfide linkages.

In some embodiments, beads comprising releasable barcode oligonucleotides may be mixed with spacer beads to adjusted or control the density of barcodes on a surface. That is, in some embodiments, spacer beads may be mixed with beads comprising releasable barcode oligonucleotides to dispose the latter on a surface in a predetermined density. In some embodiments, such mixture may comprise a ratio of spacer beads to beads comprising releasable barcode oligonucleotides of 10 percent or greater, or 25 percent or greater, or 50 percent or greater, or 80 percent or greater. The compositions and size of spacer beads may be the same or different than the composition and size of beads comprising releasable barcode oligonucleotides. In some embodiments, spacer beads have the same composition and size as beads comprising releasable barcode oligonucleotides.

In some embodiments, methods for making spatially barcoded surfaces may comprise steps of (a) providing a surface comprising capture oligonucleotides attached thereto and a plurality of closely spaced beads disposed thereon, wherein each bead comprises releasably attached barcode oligonucleotides each comprising a barcode sequence; (b) releasing the barcode oligonucleotides so that the barcode oligonucleotides are captured by capture oligonucleotides; and (c) surface amplifying the captured barcode oligonucleotides to form clonal populations of captured barcode oligonucleotides on the surface. As used herein in reference to beads on a surface, the terms “closely spaced” or “closely packed” means that a maximal number of beads are disposed on the surface for the area of the surface. In some embodiments, the terms mean that substantially every bead on the surface is contiguous with or touching at least a plurality of adjacent beads. In some embodiments, methods described herein may further comprise providing the surface with, or adding to the surface, a diffusion inhibitor which fills the interstitial spaces between the beads. In some embodiments, such diffusion inhibitor may comprise a fluid, such as an oil, immiscible with a carrier fluid used to load beads onto the surface. In other embodiments, such diffusion inhibitors may comprise soluble polymers, such as agarose, poly(ethylene glycol) (PEG), dextran, poly(vinyl) alcohol, poly(vinyl) acetate, polyamide, polysaccharide, poly(lysine), polyacrylamide, poly(ethylene oxide), poly(acrylic acid), or the like. Oils can include, but are not limited to, Fluoroinert-40 (FC-40); Fluoroinert-80 (FC-80); DuPont Krytox fluorinated oils; HFE-7500 (fluorinated oil); Perfluorodecalin; mineral oil; corn oil; soybean oil; silicone oil; and the like. In some embodiments, a diffusion inhibitor may be a viscosity modifier, such as, glycerol, hydroxyethyl cellulose, carboxymethyl cellulose, or the like. In some embodiments, a diffusion inhibitor may be a gel barrier. A gel barrier may comprise a hydrogel. In some embodiments, such a hydrogel may comprise a degradable hydrogel. In some embodiments, beads are degradable hydrogel beads and the diffusion inhibitor is a degradable hydrogel.

In some embodiments, a surface with beads disposed thereon is formed by loading a flow cell (comprising the surface) with a carrier fluid comprising the beads. In some embodiments, the carrier fluid comprises an aqueous solution, such as, a phosphate-buffered saline solution, a TE buffer, or the like. In some embodiments, such carrier fluid may also comprise one or more diffusion inhibitors.

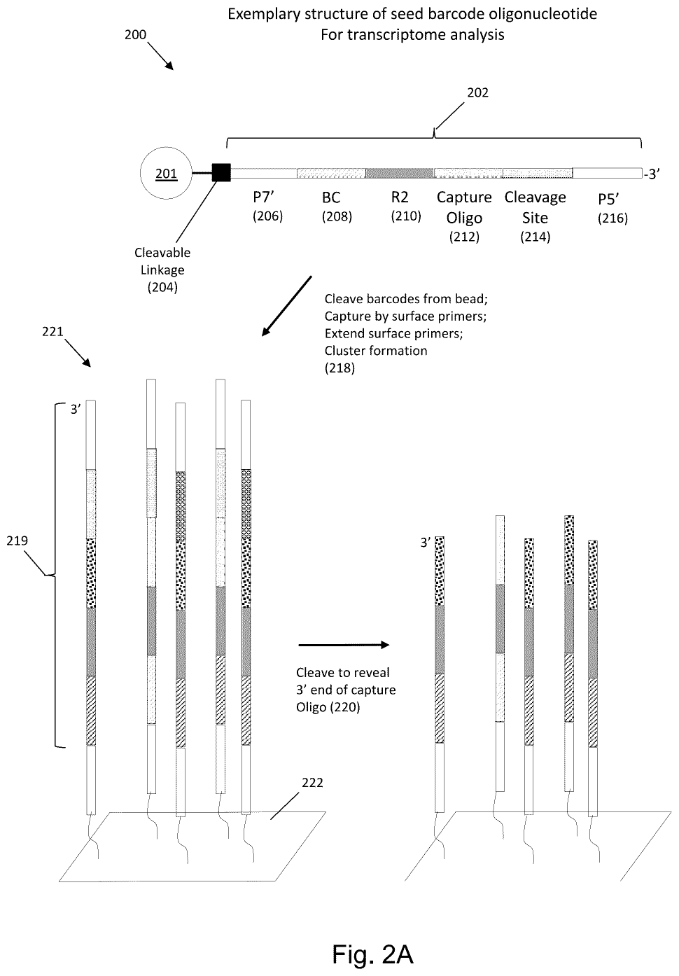

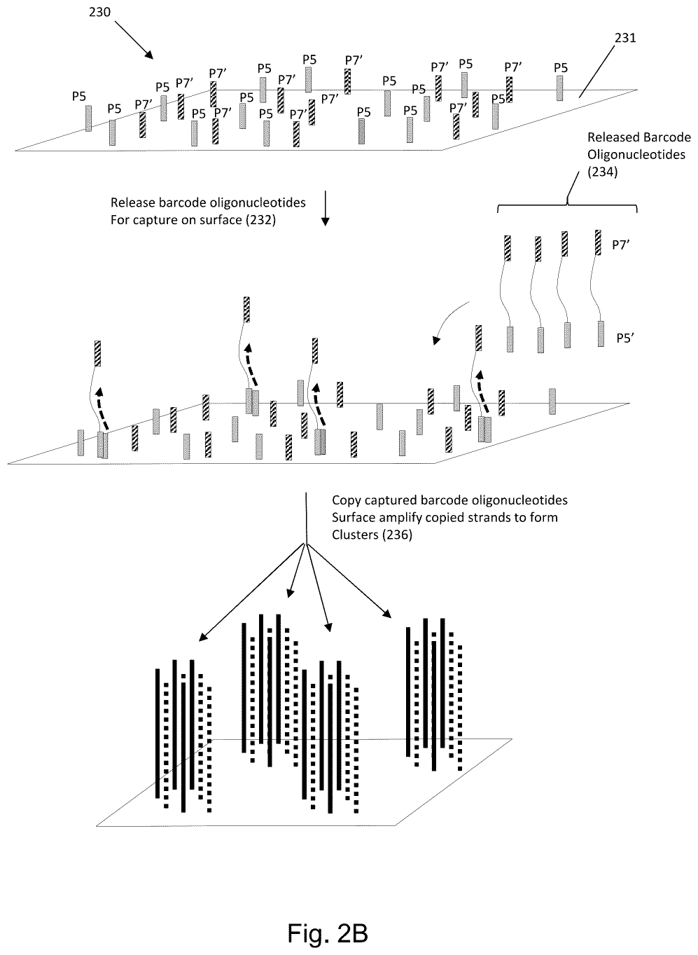

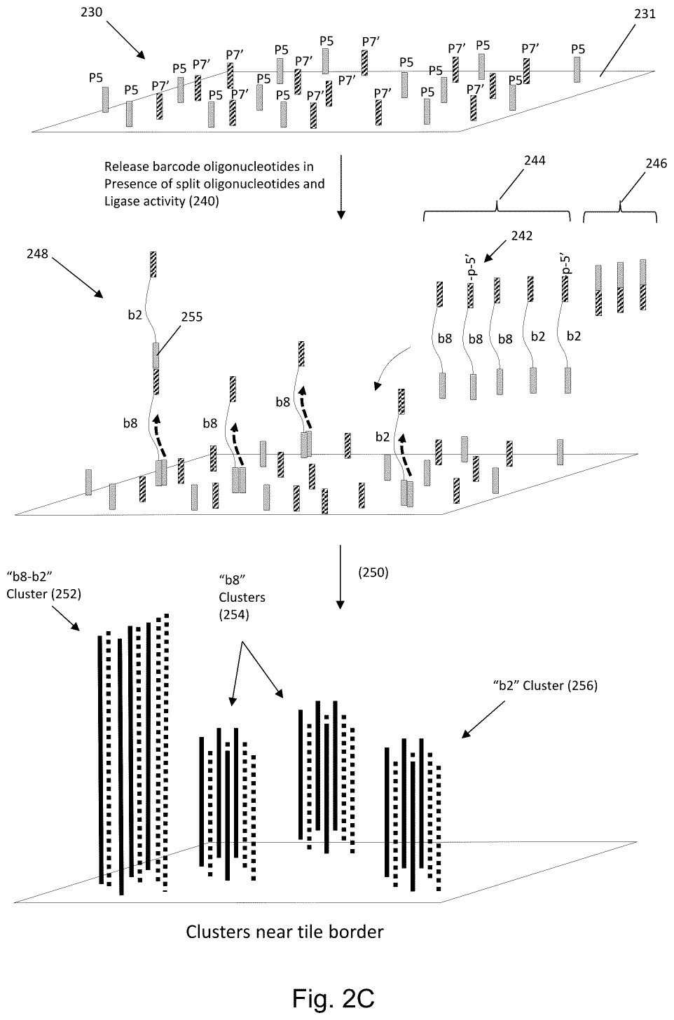

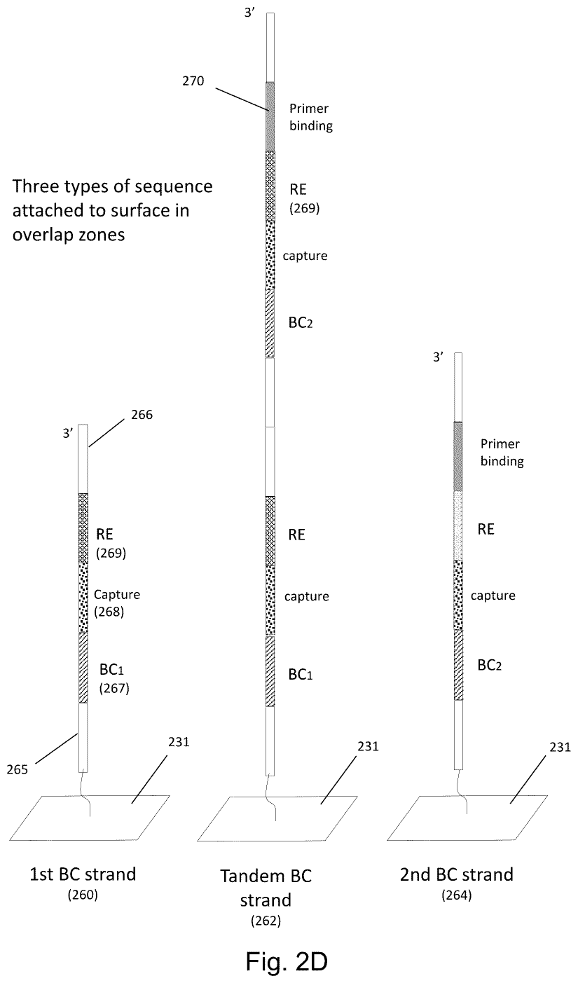

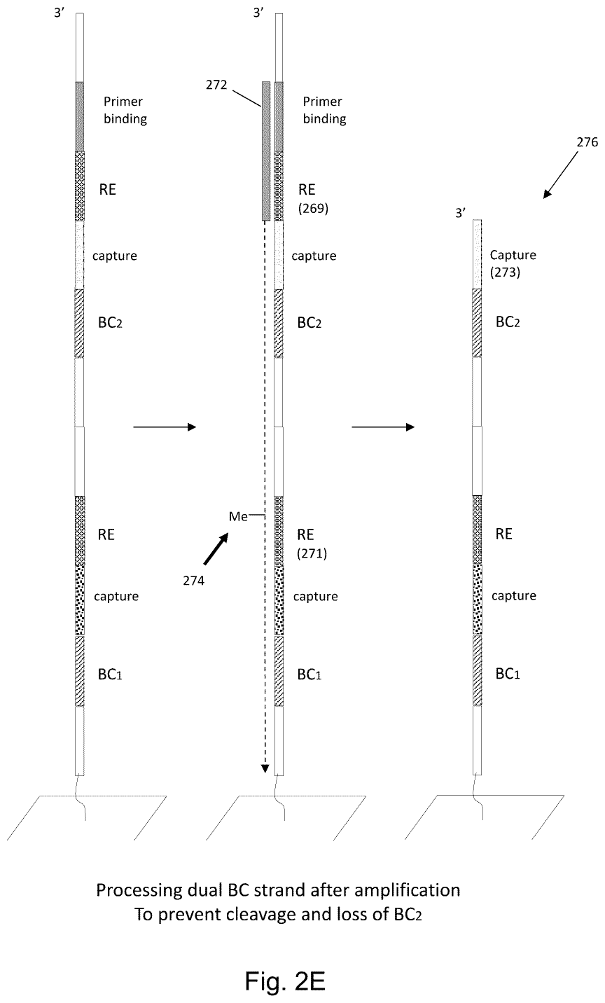

In some embodiments, barcode sequences of barcode oligonucleotides may be synthesized on beads by a split-and-pool procedure, e.g. using phosphoramidite chemistry, such as disclosed by Church, U.S. Pat. No. 4,942,124; Godron et al, International patent publication WO2020/120442; Seelig et al, U.S. patent publication 2016/0138086; and the like. Such split-and-pool procedures using all four natural nucleotides produces a random N-mer sequence having the form “—NNN . . . N—”, wherein the oligonucleotides synthesized on each different bead has the same random N-mer, or barcode, sequence. Of course, other types of barcode structures may be used with the methods described herein, e.g. Brenner, U.S. Pat. No. 5,635,400; Mao et al, International patent publication WO2002/097113; or the like. The number of nucleotides in the random-mer sequence determines the size of the set of barcode oligonucleotides; or, in other words, the number of different barcode sequences. In some embodiments, at least 10,000 different barcode sequences are employed, or at least 100,000 different barcode sequences are employed, or at least 500,000 different barcode sequences are employed, or at least 1,000,000 different barcode sequences are employed. As noted in FIG. 2A, barcode oligonucleotides may have additional elements, e.g. complementary sequences to surface capture oligonucleotides, complementary sequences to molecules of interest, such as mRNA, or the like. A wide variety of cleavable linkages may be used to releasably attached barcode oligonucleotides to beads. For example, the following references (which are incorporated by reference) disclose several suitable cleavable linkers: Leriche et al, Bioorganic & Medicinal Chemistry, 20:571-582 (2012); Urdea et al, U.S. Pat. No. 5,367,066; Monforte et al, U.S. Pat. No. 5,700,642; Glen Research application note, GR-33-11; and the like. In some embodiments, barcode oligonucleotides are releasably linked to beads by a photocleavable linkage as described in Urdea et al (cited above). In some embodiments, a photocleavable (or photo-releasable) linkage is used to attached barcode oligonucleotides to beads. In some embodiments, such photocleavable linkage comprises a nitro-benzyl group as described in Urdea et al (cited above). In some embodiments, a chemically cleavable linkage is used to attach barcode oligonucleotides to beads. In some embodiments such chemically releasable linkage is a disulfide linkage, releasable by treatment with a reducing agent, e.g. as described in U.S. patent publication US2014/0378322 and/or Glen Research application note, GR-33-11, which are incorporated herein by reference. In some embodiments, both the barcode oligonucleotides are releasable and the beads are degradable by cleavage of disulfide-containing cross-linkers.

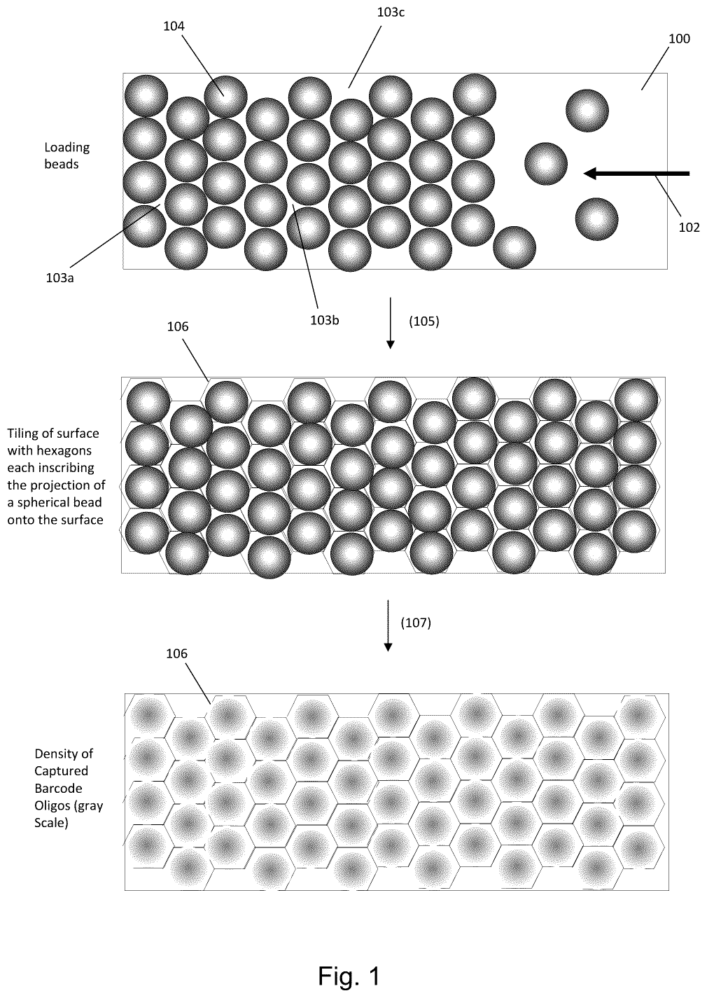

FIGS. 1 and 2A-2B illustrate an embodiment of the systems and methods described above. As shown in FIG. 1, beads (104) can be loaded (102) onto surface (100). In some embodiments, such loading may be accomplished by flowing beads suspended in a carrier fluid through a flow cell. A flow cell may comprise a variety of structures. In some embodiments, a flow cells comprises a channel having at least one planar surface. In some embodiments, the channel may have an inlet and an outlet and may be configured to retain beads so that they accumulate on the surface as the carrier fluid suspending them is passed through the channel. In some embodiments, the term “channel” means a container capable of holding fluid (which may be static or flowing) and having at least one surface on which beads may be disposed. In some embodiments, a channel may constrain a flow of fluid therethrough from an inlet to an outlet. In other embodiments, a channel may comprise a non-flowing volume of fluid that may be removed, replaced or added to by way of an opening or inlet; that is, in some embodiments, a channel may be a well or a well-like structure. A surface may be a surface, e.g. a planar surface, of a solid material, such as, glass, quartz, plastic, a metal oxide, or the like.

Returning to FIG. 1, as beads become closely packed on surface (100) regions between the beads, or interstitial spaces, such as (103a-c), form. A tiling of surface (100) may be made after surface (100) is fully packed (105), wherein each tile corresponds to a separate region of surface (100) under a bead. Such tilings assist in determining the relative positions of barcode oligonucleotides on a surface, as is described more fully below. In the case of FIG. 1, the tiles are hexagons (e.g. (106)); however, tiles of other shapes also may be employed, such as, squares or triangles. In some embodiments, a tiling of a surface covers the surface completely; that is, all areas or sub-areas of a surface are coincident with a tile area. In some embodiments, an exception to the immediately preceding sentence may be areas at the edge of a surface. In some embodiments, the areas of each tile is equal to that of every other tile. Hexagonal tiles are advantageous whenever beads are spherical because a hexagonal tiling lines up with, or corresponds to, a closely packed layer of spherical beads (i.e. a projection of a spherical bead onto the planar surface results in a disk inscribed on the interior of the hexagonal tile). After barcode oligonucleotides are released from a bead, most are captured by complementary capture oligonucleotides attached to surface (100) within the disk-like sub-region below the bead, as illustrated in the bottom panel of FIG. 1. After such capture, the barcode oligonucleotides are copied and amplified by a surface amplification technique, such as bridge PCR amplification. As used herein, “surface amplification” means a linear or exponential amplification of a polynucleotide or its complement with at least a portion of the resulting amplicon being covalently attached to the surface. In some embodiments, surface amplification requires that a surface have attached one or more capture probes which serve as primers for copying. Surface amplification techniques include, but are not limited to, bridge polymerase chain reaction (PCR), recombinase-polymerase solid phase amplification (RPA), kinetic exclusion amplification, or the like. Some surface amplification techniques are disclosed in the following references which are incorporated by reference: Adams, U.S. Pat. No. 5,641,658; Boles, U.S. Pat. No. 6,300,070; Mayer, U.S. Pat. Nos. 7,790,418, 7,985,565, 8,652,810, 9,593,328, 9,902,951 and International patent publication WO1998/44151; Ronaghi, U.S. Pat. Nos. 97,773,268, 9,416,415; 7,763,427, 8,426,134, 7,666,598, 9,309,558; or U.S. Pat. Nos. 6,090,592; 6,060,288; 6,787,308; 9,057,097; 9,169,513; 9,476,080; 9,476,080; Adessi et al, Nucleic Acids Research, 28 (20): e87 (2000); and the like. After capture, seed sequences (i.e. barcode oligonucleotides released from beads) may be copied and amplified to produce larger numbers of surface-bound copies using conventional surface amplification techniques, such as, bridge amplification or the like. In some embodiments, clusters are produced from the captured barcode oligonucleotides by bridge amplification. Such embodiments may further comprise cleaving the ends of the amplified sequences distal to the surface to expose extendable capture sequences.

After such surface amplification, clusters of barcode oligonculeotides are processed, e.g. by enzymatic cleavage, to give a barcoded surface. Such surface may be employed as is, although the identity of the barcode sequences in the various tiles is unknown. Such barcoded surfaces may be used to indicate proximity of events or compounds, for example, messenger RNAs (mRNA) derived from the same cell that may be occupying a given tile.

In some embodiments, a diffusion inhibitor may be placed in the interstitial spaces between the beads prior to the release of barcode oligonucleotides. In this way, the amounts of barcode oligonucleotides that diffuse to adjacent tiles is minimized or eliminated. In some embodiments, such mixing of barcode oligonucleotides on the surface in the border, or edge, regions of the tiles is undesirable because it reduces the accuracy of a spatial determinations using the barcode sequences. In some embodiments, such mixing of barcode oligonucleotides at the border or edge regions of tiles can be used advantageously to determine the relative positions of the tiles, as is discussed more fully below. In some embodiments, a bead carrier fluid may comprise a diffusion inhibitor.

FIG. 2A illustrates an example of a structure of an oligonucleotide attached to a bead or particle. Bead-oligonucleotide conjugate (200) comprises bead (201) and oligonucleotide (202) cleavably attached to bead (201), for example, by cleavable linkage (204) or by hybridization to an oligonucleotide attached to bead (201). In some embodiments, oligonucleotide (202) comprises a plurality of segments including, but not limited to, segments at either end for surface amplification (shown as P7 (206) and P5 (216)), a segment with one or more barcodes (208), a primer binding segment (210) for sequencing all or a portion of barcode (208), a capture segment (212), and cleavage site (214) for removing the amplification segment (e.g. P5) so that the end of the capture segment (212) may be extended. Bead or particle (201) may vary widely in regard to size, composition, and loading with oligonucleotide (202). In some embodiments, the amount (or loading) of oligonucleotide (202) on bead (201) is sufficient to dispose on the surface bounded by a chamber oligonucleotides (202) in a density in the range of 0.5 to 2 oligonucleotides per 1 μm². Oligonucleotides (202) are released by cleaving cleavable linkage (204), oligonucleotides (202) are captured on surface (222) by complementary surface-bound primers, such primers are extended copying oligonucleotide (202), and extension products (219) are amplified to form clusters (221). After formation of clusters of double stranded oligonucleotides, segments containing the P5 primer binding site may be cleaved (220). For example, whenever capture oligonucleotide (212) is a polyT segment for capturing mRNA, cleavage site (214) may be a DraI endonuclease recognition site.

FIG. 2B illustrates the above process. Surface (231) has attached a “lawn” (230) of two kinds of primers, designated P5 and P7 (i.e. capture oligonucleotides), attached to surface (231) by their 5′ ends. Barcode oligonucleotides (234) are released or cleaved (232) from beads and are captured by the P5 capture oligonucleotides, after which they are copied by extending the P5 primers (in the presence of a suitable DNA polymerase and dNTPs). After such copying, the extension products are surface amplified to give clusters (236).

As mentioned above, in some embodiments, barcode oligonucleotides may be released under conditions wherein barcode oligonucleotides not only “leak” or diffuse to adjacent tiles, but also under conditions wherein such “leaking” barcode oligonucleotides may be ligated to barcode oligonucleotides comprising different barcode sequences. Such ligated barcode oligonucleotides are referred to herein as “tandem barcode oligonucleotides,” since they comprise two or more barcode sequences, at least two of which come from adjacent tiles. Tandem barcode oligonucleotides may be used to determine the relative positions of tiles on a surface. This is advantageous because the barcodes need be sequenced only once (e.g. after being integrated into a cDNA), rather than twice as is the case with many current spatial barcode methods, e.g. Chen et al, Cell, 185:1777-1792 (2022); Cho et al, bioRxiv (https://boi.org/10.1101/2021.01.25.427807); Fu et al, Cell, 185:4621-4633 (2022); Rodriques et al, Science, 363 (6434): 1463-1467 (2019); and the like. In some embodiments, these conditions are achieved by delivering to a surface for reaction with barcode oligonucleotides one or more splint oligonucleotides and a ligase activity. A portion of barcode oligonucleotides released from each bead may comprise a 5′ phosphate group that whenever brought into juxtaposition with a 3′ hydroxyl of a barcode oligonucleotide by hybridization to a splint oligonucleotide a phosphodiester bond is formed covalently linking the barcode oligonucleotides. In some cases, self-ligation could occur, but such side reactions will not affect the method and can be compensated for by adjusting the portion of barcode oligonucleotides comprising 5′ phosphate groups, for example, by increasing the portion. In some embodiments, the portion of barcode oligonucleotides comprising a 5′-phosphate group is in the range of from 0.5 percent to 10 percent; in other embodiments, the portion of barcode oligonucleotides comprising a 5′-phosphate group is in the range of from 1 to 5 percent. These percentages may be conveniently obtained when using the nitrobenzyl photocleavable linkers of Urdea et al (cited above) as the position of the nitrobenzyl moiety in the linker may be selected to produce either a 5′-phosphate or a 5′-hydroxyl. Thus, the two forms of the linker may be used in a proportion corresponding to the desired fraction of barcode oligonucleotides having 5′-phosphate groups. The embodiment of FIG. 2C starts with the same surface (231) and lawn of capture oligonucleotides as the embodiment of FIG. 2B. Barcode oligonucleotides (244), a portion thereof comprising 5′-phosphate groups (242), are released (240) in the presence of splint oligonucleotides (246) on or near surface (231), so that at least some barcode oligonucleotides from adjacent tiles are ligated to form tandem barcode oligonucleotides (248). In some embodiments, the splint oligonucleotides may be rendered non-ligatable by the presence of a 3′ blocking group or a dideoxynucleotide at their 3′ ends. In some cases, a tandem barcode oligonucleotide may be captured by one of the ligated primer binding sites (255), but like the circularized barcode oligonucleotides, and ligation of 5′ phosphorylated barcode strands directly to surface primers, such side products will not matter since the desired capture configurations (as shown in FIG. 2C) may be increased or decreased by adjusting reaction conditions. After capture and extension of barcode oligonucleotides (244) and any tandem barcode oligonucleotides formed therefrom, clusters of clonal oligonucleotides comprising barcode sequences are formed by surface amplification (250). In the border or edge regions of adjacent tiles on surface (231) clusters containing a variety of barcode sequences are formed. For example, as illustrated in FIG. 2C, some cluster may comprise tandem barcode sequences (252), such as b2 and b8, while other cluster may comprise single barcode sequences (e.g. (254) and (256)) comprising b8 solely and b2 solely, respectively.

FIG. 2D illustrates embodiments of barcode-containing oligonucleotides that may be formed near the borders of adjacent tiles. At least three different barcode-containing oligonucleotides may be formed: first barcode strands (260), tandem barcode strands (262), and second barcode strands (264). In some embodiments, as described above, oligonucleotide strands containing barcode sequences comprise primer (265) attached by its 5′ end to surface (231) and primer (266) with a free 3′ hydroxyl to permit solid phase amplification, barcode sequence BC1 (267), capture sequence (268) (which for transcriptome analysis may be a polyT segment), and restriction endonuclease site RE (269) (which is used to cleave the distal portion of the oligonucleotide strand so that a free 3′ end of the capture oligonucleotide is produced). A strand from an adjacent tile may further include primer binding site (270) whenever restriction digestion is employed to produce a capture sequence, that may be extended. The restriction sites of the first barcode oligonucleotides and the second barcode oligonucleotides may be the same or different. In some embodiments, the first barcode oligonucleotides may comprise a restriction site for a methylation sensitive endonuclease; that is, an endonuclease that is prevented from cleaving whenever its recognition site is methylated in a specific manner. With such an arrangement, cleavage at only the distal restriction site of tandem barcode strands (262) may be accomplished, as illustrated in FIG. 2E. A portion of the sequence of primer (272) is complementary to recognition site (269), so that when annealed to tandem barcode oligonucleotides after amplification a non-methylated restriction site is formed. Primer (272) is then extended with a polymerase in the presence of the four deoxynucleoside triphosphates (dNTPs) with one of the dNTPs substituted with an appropriately methylated analog. Restriction sites in the direction of surface (231) from restriction site (269) (such as restriction site (271)) can be methylated. In some embodiments, type IIs endonuclease, Awl I (New England Biolabs) may be employed for this purpose. In this case, primer (272) would be extended with dATP substituted by the analog N6-methyldeoxyadenosine 5′-triphosphate (which may be prepared as described in Mace, J. Biol. Chem. 259 (6): 3616-3669 (1984); and/or Jones et al, J. Am. Chem. Soc., 85 (2): 193-201 (1963)). After such extension and digestion, the extension product and the cleaved distal segments can be washed away to give tandem barcode sequence (276) with distal capture sequence (273) exposed.

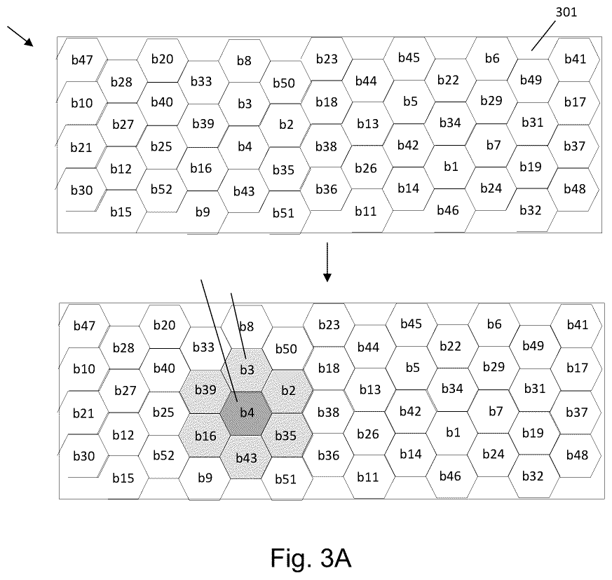

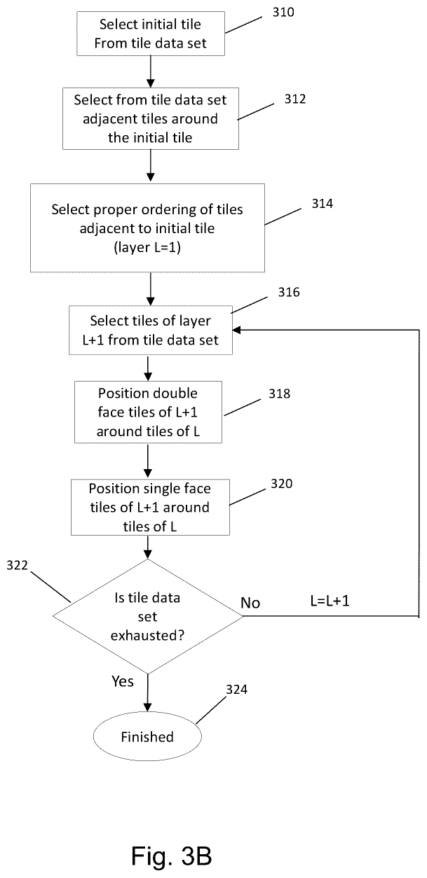

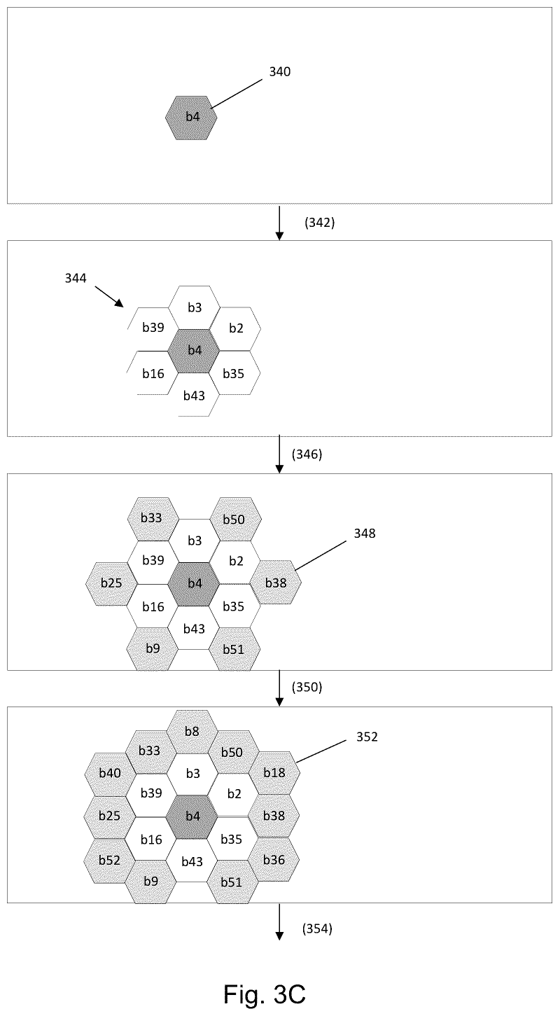

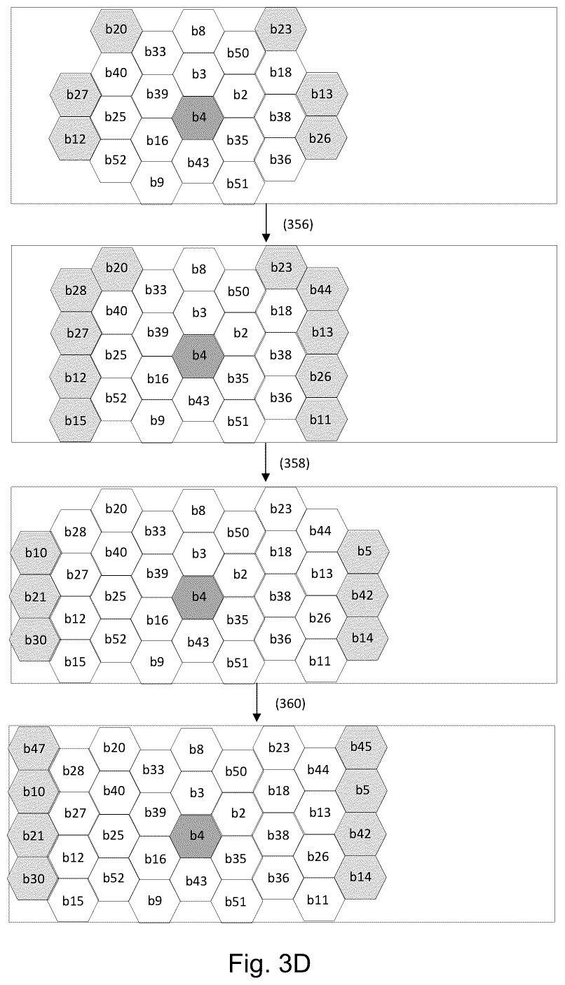

The relative locations of tiles on a surface may be determined using the tandem barcode sequences, for example, using the algorithm illustrated in FIGS. 3A-3D. In other embodiments, other algorithms may be employed. The algorithm of FIGS. 3A-3D is derived by noticing properties of a solution (e.g. (300)), assuming (i) same-sized spherical beads are closely packed together on a surface so that each bead is centered in a hexagon of a hexagonal tiling of the surface, (ii) barcode oligonucleotides of each bead carry the same unique barcode sequence, (iii) a given percentage of barcode oligonucleotides from any selected tile diffuse to each adjacent tile, and (iv) released barcode oligonucleotides do not diffuse any further than an adjacent tile (or do so in only negligible amounts). One of ordinary skill in the art would recognize that deviations from such assumptions may require additional conventional algorithmic features and/or data analysis in order to determine relative positions of tiles. For example, the step of loading beads on a surface may result in a distribution of beads that may be associated with a quasi-tiling because of gaps or aggregation behaviors of the beads. In some embodiments, the barcode sequences in each tile may be divided between a primary barcode sequence (e.g. b4 (304) in FIG. 3A) and a plurality up to six secondary barcode sequences (e.g. b39 (306) in FIG. 3A and as illustrated in Table 1). The upper panel of FIG. 3A shows primary barcode designations (b1 to b52) distributed to the hexagonal tiles. That is, the primary barcode designations indicate where the beads carrying the indicated barcode sequences resided on surface (301) at the time barcode oligonucleotides were released. If any tile is selected, for example, tile b4 (304), the adjacent tiles to b4 are immediately discernible by examining sequences of tandem barcodes containing the b4 barcode sequence. The non-b4 barcode sequences (of the tandem barcodes) indicate the barcode sequences of the barcode oligonucleotides that were released in the tiles adjacent to the b4 tile. The ordering of the adjacent tiles around the b4 tile can be determined by examining the sequences of the tandem barcode oligonucleotides in each of the adjacent tiles. For example, tile b3 (306) will contain tandem barcode oligonucleotides with b3-b2 sequences and b3-b39 sequences, but not b3-b43 sequences. Thus, by examining the tandem barcode sequences associated each primary barcode sequence, adjacent tiles of each tile can be determined. Primary barcode sequences are discerned by the frequency of their occurrence, such as illustrated in Table 1. For example, if a spatial distribution of transcriptomes is being measured, sequence data will show a dominate fraction of mRNA sequences linked with single barcode sequences and only a minor fraction linked with tandem barcodes (e.g. 82% versus 18% or less in the example of Table 1).

TABLE 1
Barcode composition in b4 tile and each adjacent tile assuming
a 3% “leakage” of barcodes to adjacent tiles

| Tile | Barcode Composition |
|---|---|
| b4 tile → | 82% b4 + 3% b3 + 3% b2 + 3% b35 + 3% b43 + 3% b16 + |
| | 3% b39* |
| b3 tile → | 82% b3 + 3% b8 + 3% b50 + 3% b2 + 3% b4 + 3% b39 + |
| | 3% b33 |
| b2 tile → | 82% b2 + 3% b50 + 3% b18 + 3% b38 + 3% b35 + 3% b4 + |
| | 3% b3 |
| b35 tile → | 82% b35 + 3% b2 + 3% b38 + 3% b35 + 3% b51 + 3% b43 + |
| | 3% b4 |
| b43 tile → | 85% b43 + 3% b4 + 3% b35 + 3% b51 + 3% b9 + 3% b16 |
| b16 tile → | 82% b16 + 3% b4 + 3% b43 + 3% b9 + 3% b52 + 3% b25 + |
| | 3% b39 |
| b39 tile → | 82% b39 + 3% b33 + 3% b3 + 3% b4 + 3% b16 + 3% b25 + |
| | 3% b40 |

FIG. 3B shows a flow chart that summarized the steps illustrated in FIG. 3A and FIGS. 3C-3D. Prior to implementing the algorithm a tile data set can be assembled which is a record of each primary barcode sequence and its associated tandem barcode sequences. The first step (310) of the algorithm represented by the flow chart of FIG. 3B can be used to select an arbitrary initial tile; that is, a tile corresponding to the arbitrarily selected primary barcode sequence (such as b4 described above). Once a primary barcode is selected from the tile data set, it can be removed. That is, whenever one or more tiles are selected for assembling a tiling by the algorithm, they can be removed from the tile data set, so that the tile data set continually shrinks as tiles are selected, until it is finally emptied. After an initial tile is selected, adjacent tiles can be selected (312) as described above (namely, by examining secondary barcodes of the tiles in the tile data set to find the ones containing tandem barcodes wherein one barcode of the tandem is the same as that of the initial tile). A proper ordering of the adjacent tile (314) can be accomplished by examining the tandem barcodes and determining that tiles only share an edge in the assembly if they each have tandem barcode sequences with the other's primary barcode sequence. After such layer of adjacent tiles are properly assembled, tiles of the next layer can be selected (316) from the tile data set. In some embodiments of the algorithm, adjacent tiles may be assembled in two stages: first (318), tiles sharing two edges with the current assembly (i.e. partially completed tiling) are examined for proper ordering with the current assembly, after which (320) tiles sharing a single edge with the current assembly and two edges with previously assembled tiles are assembled (in a proper ordering). After such assembly, the algorithm determines is the tile data set is empty (322). If yes, then the assembly is complete (324); if not, then another selection can be made for layer L+1 in accordance with step (316).

Steps of the flow chart of FIG. 3B are illustrated in FIGS. 3C-3D. First tile (340) can be selected, after which (342) adjacent tiles are selected and assembled around the initial tile (344). The 2-edge tiles (e.g. (348) of the next layer can then be selected from the tile data set and properly assembled with the previous layer, after which (350) 1-edge tiles (e.g. 352) are properly assembled. The process can continue similarly (354)-(360) until the tile data set is exhausted.

One of ordinary skill would recognize that information about the distribution of beads on a surface derived from an image may be used in determining relative positions of tiles. For example, locations, sizes and shapes of sub-regions without beads (but large enough to be occupied with one or more beads) may be used in determining whether or not particular tandem barcode oligonucleotides should be present in a tile or not. Likewise, locations, sizes and shapes of gap between beads may be used in the same way.

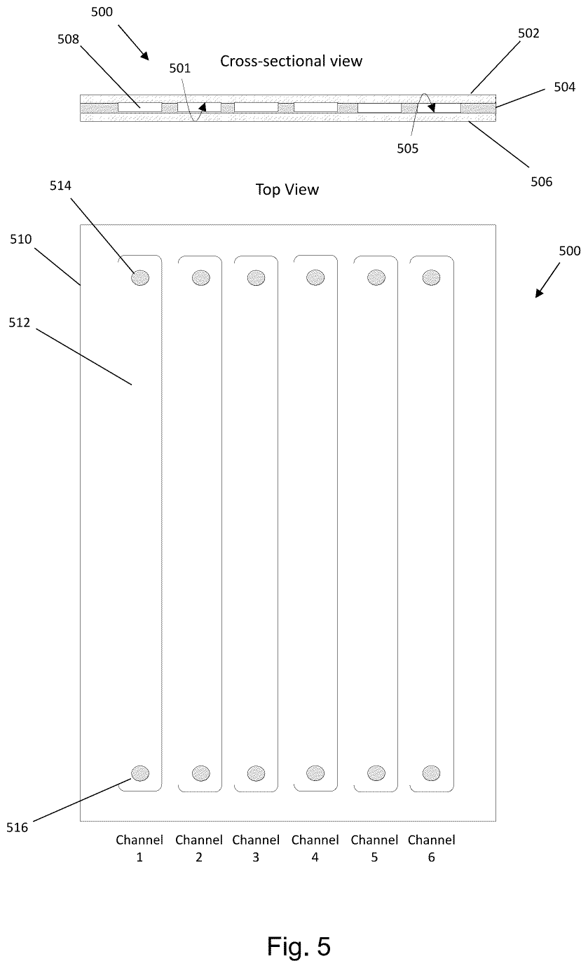

In some embodiments, a plurality of channels, each with at least one surface, may be arranged together in a flow cell as illustrated in FIG. 5. In some embodiments, the plurality of channels may be in the range of from 2 to 12, or from 2 to 8, or from 2 to 6, or in the range of from 2 to 4. A flow cell (500) is shown in a cross-sectional view and a top view. Flow cell (500) has bottom, or first, wall (506) with first surface (505); top, or second, wall (502) with second surface (501); and sandwiched sealingly therebetween spacer (504) whose longitudinal holes form channels 1-6, one of which is indicated by (508) in the cross-sectional view, and by (512) in the top view. In some embodiments, spacer (504) may have a thickness in the range of from 10 μm to 500 μm, or in the range of from 50 μm to 250 μm, which determines the interior height of the channels. Top wall (502) comprises inlets (514) and outlets (516) for either separately or jointly loading and removing reagents and beads from channels 1-6. In some embodiments, at least one of walls (502) and (506) are made of light transmissive materials, such as glass, plastic, or the like. Flow cell (500) may be operationally associated with a fluidic device that delivers reagents and beads to any of channels 1-6 under programmed control. Guidance for particular designs, including fluid handling and valving for such fluidic systems may be found in U.S. Pat. Nos. 8,921,073; 8,173,080; 8,900,828; and the like, which are incorporated herein by reference.

As noted above, any of first surfaces, second surfaces or polymer matrix wall of chambers may comprise capture elements and other functional groups for carrying out a variety of operations including, but not limited to, capturing beads, capturing analytes (such as, mRNA, secreted proteins, intracellular proteins, or genomic sequences), capturing constituents of analytical reagents (such as, oligonucleotide labels from antibodies), and the like. Derivatizing surfaces for such purposes is well-known to those skilled in the art, as evidenced by the following references: Hermanson (cited above); and the like. As noted above, in some embodiments, a fluidic device of the method comprises or may be operationally associated with a detector (e.g. a microscope) that permits beads in a channel to be imaged.

### Hydrogel Beads

A wide variety of degradable and non-degradable hydrogel beads may be used with the systems and methods described herein. Spherical beads of predetermined sizes may be made using known techniques, e.g. Hinz et al, U.S. Pat. No. 9,249,461; Light et al, U.S. patent publication 2015/0175734; Weaver et al., U.S. Pat. No. 5,055,390; Tmovsky et al., U.S. Pat. No. 6,586,176; Hindson et al, U.S. patent publication US2014/0378322; and the like.

In some embodiments, a channel of a fluidic device comprises one or more polymer precursors for forming chambers. In some embodiments, the one or more polymer precursors comprise hydrogel precursors. Such precursors may be selected from a wide variety of compounds including, but not limited to, polyethylene glycol (PEG)-thiol, PEG-acrylate, acrylamide, N,N′-bis(acryloyl) cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or combinations or mixtures thereof. In some embodiments, the hydrogel comprises an enzymatically degradable hydrogel, PEGthiol/PEG-acrylate, acrylamide/N,N′-bis(acryloyl) cystamine (BACy), or PEG/PPO. In some embodiments, the following precursors and crosslinker may be used to form chambers with degradable polymer matrix (hydrogel) walls.

Polymer precursors may be formed by using any hydrogel precursor and crosslinkers of Table 2 (columns 1 and 3, respectively). The resulting polymer matrices may be degraded with the indicated degradation agents in Table 2 (column 4).

TABLE 2
Examples of precursors, hydrogels, crosslinkers,
and degradation agents

| | | | Degradation |
|---|---|---|---|
| Precursors | Hydrogels | Crosslinkers | Agents |
| Acrylamide | Polyacrylamide | Bis-acryloyl cystamine | DTT/TCEP/ |
| | | (structure 1) | THP |
| PEG-based | PEG | Bis(2- | DTT/TCEP/ |
| acryloyl | | methacryloly)oxyethyl | THP |
| | | disulfide (structure 2) | |
| Dextran-based | Dextran | N,N′-(1,2- | NaIO4 |
| acryloyl | | Dihydroxyl- | |
| | | ethylene)bis- | |
| | | acrylamide(structure 3) | |
| Polysaccharide- | Polysaccharide | Structure 4 | NaOH, |
| base acryloyl | | | ethanolamine |
| | | | DTT/TCEP/ |
| | | | THP |
| Gelatin-base | Gelatin | Structure 5 | NaOH, |
| acryloyl | | | ethanolamine, |
| | | | nucleophilic |
| | | | bases |
| | | Structure 6 | NaOH, alkali, |
| | | | organic bases |
| | | Structure 7 | Acid |

TABLE 3
Structures of Example Crosslinkers

| | |
|---|---|
| Structure #1 | 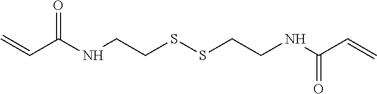 [SMILES: C=CC(=O)NCCSSCCNC(=O)C=C] |
| Structure #2 | 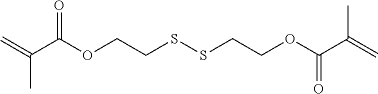 [SMILES: C=C(C)C(=O)OCCSSCCOC(=O)C(=C)C] |
| Structure #3 | 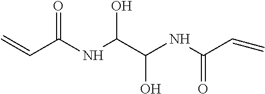 |
| Structure #4 | 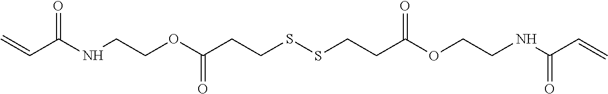 [SMILES: C=CC(=O)NCCOC(=O)CCSSCCC(=O)OCCNC(=O)C=C] |
| Structure #5 | 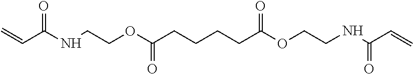 [SMILES: C=CC(=O)NCCOC(=O)CCCCC(=O)OCCNC(=O)C=C] |
| Structure #6 | 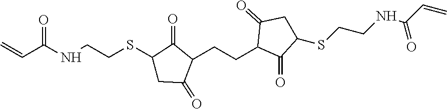 [SMILES: C=CC(=O)NCCSC1CC(=O)C(CCC2C(=O)CC(SCCNC(=O)C=C)C2=O)C1=O] |
| Structure #7 | 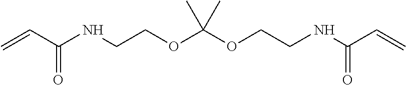 [SMILES: C=CC(=O)NCCOC(C)(C)OCCNC(=O)C=C] |

In some embodiments, hydrogel beads are degradable or depolymerizable. Hydrogel beads that are generally degradable can be degraded by treatment with a degradation agent, or equivalently, a depolymerization agent that is exposed to all beads. Depolymerization agents may include, but are not limited to, heat, light, and/or chemical depolymerization reagents (also sometimes referred to a cleaving reagents or degradation reagents). In some embodiments, degradation may be implemented using polymer precursors that permit photo-crosslinking and photo-degradation, for example, using different wavelengths for crosslinking and for degradation. For example, Eosin Y may be used for radical polymerization at defined regions using 500 nm wavelength, after which illumination at 380 nm can be used to cleave the cross linker. In other embodiments, photo-caged hydrogel cleaving reagents may be included in the formation of polymer matrix walls. For example, acid labile crosslinkers (such as esters, or the like) can be used to create the hydrogel and then UV light can be used to generate local acidic conditions which, in turn, degrades the hydrogel. In some embodiments, hydrogel beads are degradable by at least one of: (i) contacting the polymer matrices of such beads with a cleaving reagent; (ii) heating the polymer matrices of such beads to at least 90° C.; or (iii) exposing the polymer matrices of such beads to a wavelength of light that cleaves a photo-cleavable cross linker that cross links the polymer of the polymer matrix.

In some embodiments, the cleaving reagent comprises a reducing agent, an oxidative agent, an enzyme, a pH based cleaving reagent, or a combination thereof. In some embodiments, the cleaving reagent comprises dithiothreitol (DTT), tris(2-carboxyethyl) phosphine (TCEP), tris(3-hydroxypropyl)phosphine (THP), or a combination thereof.

### Applications

After spatial barcodes are established on a surface, oligonucleotide labels, barcodes, genomic fragments, messenger RNAs and similar polynucleotide targets may be sequenced by methods and systems described herein. In some embodiments, capture elements for this purpose include oligonucleotides attached along with barcode oligonucleotides to a surface, for example, in a channel, wherein such oligonucleotides comprise a sequence segment that is complementary to that of the nucleic acids to be captured, which may be polyA segments of mRNAs or an arbitrary “handle” sequence region adjacent to a barcode or oligonucleotide label. A spatial barcode provides channel or surface position information, and permits externally determined sequences to be associated with spatial locations on the surface. In some embodiments, the preparation of polynucleotides for a sequencing operation takes place after the target templates (e.g. oligonucleotide label, mRNAs, genomic fragments) are released from cells and captured by complementary sequences in the barcode oligonucleotides of clusters. In other embodiments, such target templates may be released from a tissue slice. A releasing step depends on the nature of the target templates. For example, oligonucleotide labels attached to antibodies by a disulfide linkage may be released by a reducing agent (which may be the same as a lysing reagent). mRNAs may be release by treating cells with conventional lysing agents. Releasing genomic fragments may require lysing and pre-amplification steps. Lysing conditions may vary widely and may be based on the action of heat, detergent, protease, alkaline, or combinations of such factors. The following references provide guidance for selection of lysing reagents, or lysing buffers, for single-cell lysing conditions for mRNA and/or genomic DNA: Thronhill et al, Prenatal Diagnosis, 21:490-497 (2001); Kim et al, Fertility and Sterility, 92:814-818 (2009); Spencer et al, ISME Journal, 10:427-436 (2016); Tamminen et al, Frontiers Microbiol. Methods, 6: article 195 (2015); and the like. Lysis conditions may include the following: 1) cells in H₂O at 96° C. for 15 min, followed by 15 min at 10° C.; 2) 200 mM KOH, 50 mM dithiotheitol, heat to 65° C. for 10 min; 3) for 4 μL protease-based lysis buffer: 1 μL of 17 μM SDS combined with 3 μL of 125 μg/mL proteinase K, followed by incubation at 37° C. for 60 min, then 95° C. for 15 min (to inactivate the proteinase K); 4) for 10 μL of a detergent-based lysis buffer: 2 μL H₂O, 2 μL 10 mM EDTA, 2 μL 250 mM dithiothreitol, 2 μL 0.5% N-laurylsarcosin salt solution; 5) 200 mM Tris pH7.5, 20 mM EDTA, 2% sarcoyl, 6% Ficoll.

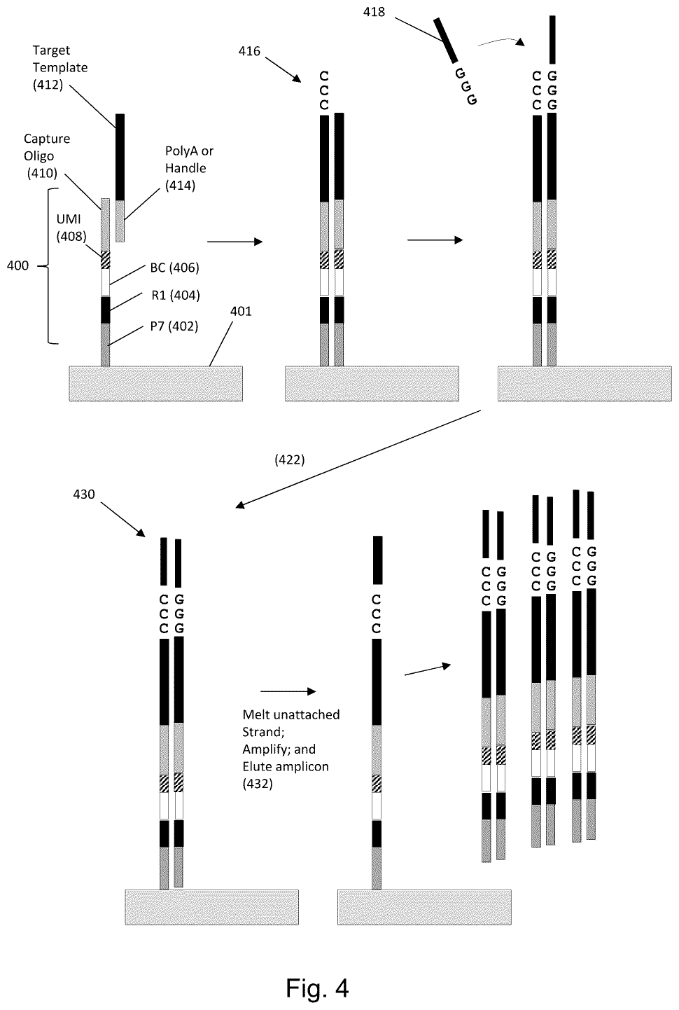

FIG. 4 illustrates a process for capturing a target template and preparing cDNAs for external sequencing. One skilled in the art would recognize that the details of the following examples of target template capture and cDNA synthesis may vary widely depending on the sequencing system employed. In some embodiments, preparation of cDNAs includes a tagmentation step. Guidance for particular embodiments may be found in Picelli et al, Genome Research, 24:2033-2040 (2014); Bose et al, Genome Biology, 16:120 (2015); Hashimshony et al, Genome Biology, 17:77 (2016); Yuan et al, Scientific Reports, 6:33883 (2016); and like references. Attached to surface (401) by their 5′ ends are oligonucleotides with the following components: primer binding site P7 (for Illumina sequencers) (402), optional primer binding site R1 (for Illumina paired end sequencing), barcode oligonucleotide (406) (which may be or include a spatial barcode), optional unique molecular identifier (408), and capture oligonucleotide (410), which may be a polyT segment whenever mRNA is to be captured. Target template (412) is captured by the hybridization of polyA segment or sequence handle (414) to capture oligonucleotide (410). After capture, capture oligonucleotide (410) and polyA segment (414) are extended by a polymerase (e.g. Moloney murine leukemia virus (MMLV) reverse transcriptase) that leaves a single stranded polyC tail (416). In some embodiments, template switching oligonucleotide (418) is hybridized thereto and the polyC tail is further extended, as show in (430), e.g. Zhu et al, Biotechniques, 30:892-897 (2001). The unattached strand is melted, the attached strand is amplified, e.g. by a PCR, and eluted for external sequencing (432).

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6^{th} edition (Saunders, 2007).

“Assay” as generally used herein refers to a process for detecting or measuring a cellular characteristic or property of single cells or of a population of cells. Typically process steps of an assay comprise a chemical, biochemical or molecular reaction (such as a cleavage of a bond, specific binding of complementary components, enzyme reactions, dissolution of complementary components, or the like) or a change of physical state (such as an increase or decrease in temperature, change in energy level, or the like) and result in the generation of a signal (or signals) from which the presence, absence or magnitude of a quantity related to a cell may be inferred. The nature of the signal produced by an assay may vary widely and can include, but is not limited to, an electrical signal, an optical signal, a chemical signal, or a material signal. A material signal comprises the production of a material that comprises information that can be extracted. For example, a material signal may be the amplification of a polynucleotide whose length, quantity, composition, or nucleotide sequence is indicative of a cellular characteristic. For example, a barcode oligonucleotide may be a material signal. Characteristics or properties of cells that are detected or measured may vary widely and include, but are not limited to, cytotoxicity, viability, proliferation capacity under selected conditions, size, shape, motility, types and profiles of cell surface, or cell membrane proteins, types and profiles of secreted proteins, production of metabolites, transcriptome, gene copy numbers, gene or allele identity, chromatin accessibility profiles, vector copy numbers for engineered or infected cells, and the like.

“Barcode” as generally used herein means a molecular label or identifier. In some embodiments, a barcode is a molecule attached to an analyte or a segment of an analyte (for example, in the case of polynucleotide barcodes and analytes) which may be used to identify the analyte. In some embodiments, a barcode (referred to herein as a “spatial barcode”) may be attached to a surface to identify a location on the surface. In some embodiments, populations of identical spatial barcodes may be disposed within a particular area on a surface. In some embodiments, there may be a one-to-one correspondence between different spatial barcodes and different areas on a surface; that is, each different area has a different and unique barcode. In some embodiments, the identity of a spatial barcode is determinable, for example, by sequencing whenever a spatial barcode is a polynucleotide. In some embodiments, a spatial barcode is an oligonucleotide. In some embodiments, an oligonucleotide spatial barcode comprises a random sequence oligonucleotide. A random sequence oligonucleotide is typically synthesized by a “split and mix” synthesis techniques, for example, as described in the following references that are incorporated herein by reference: Church, U.S. Pat. No. 4,942,124; Godron et al, International patent publication WO2020/120442; Seelig et al, U.S. patent publication 2016/0138086; and the like. Sometimes a random oligonucleotide is represented as “NNN . . . N.” In some embodiments, the term “barcode” includes composite barcodes; that is, an oligonucleotide segment that comprises sub-segments that identify different objects. For example, a first segment of a composite barcode may identify a particular area of a surface and a second segment of a composite barcode may identify a particular molecule (a so-called “unique molecular identifier” or UMI).

“Cleavable linkage” or “cleavable nucleotide” as generally used herein means any of wide variety of cleavable linkages, or more particularly, cleavable nucleotides, may be used with embodiments described herein. As used herein, the term “cleavable site” refers to a nucleotide or backbone linkage of a single stranded nucleic acid sequence that can be excised or cleaved under predetermined conditions, thereby separating the single stranded nucleic acid sequence into two parts. In some embodiments, a step of cleaving a cleavable nucleotide or a cleavable linkage leaves a free 3′-hydroxyl on a cleaved strand, thereby, for example permitting the cleaved strand to be extended by a polymerase. Cleaving steps may be carried out chemically, thermally, enzymatically or by light-based cleavage. Sometimes the term “releasing” may be used in reference to cleaving an oligonucleotide label, for example, by a releasing reagent or agent, which may be one or more of those listed above. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage by glycosylases and/or endonucleases may require a double stranded DNA substrate. Methods synthesizing and cleaving nucleic acids containing chemically cleavable, thermally cleavable, and photo-labile groups are described for example, in U.S. Pat. No. 5,700,642, which is incorporated herein by reference. Further cleavable linkages are disclosed in the following references: Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728, Urdea et al, U.S. Pat. No. 5,367,066, which are incorporated herein by reference. Synthesis and cleavage conditions of chemically cleavable oligonucleotides are described in U.S. Pat. Nos. 5,700,642 and 5,830,655. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide. In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide, which may (if desired) be removed by a phosphatase. In some embodiments, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al., J. Org. Chem. 61:525-529 (1996), Kahl et al., J. Org. Chem. 64:507-510 (1999), Kahl et al., J. Org. Chem. 63:4870-4871 (1998), Greenberg et al., J. Org. Chem. 59:746-753 (1994), Holmes et al., J. Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially. In some embodiments, ribonucleotides may be employed as cleavable nucleotides, wherein a cleavage step may be implemented using a ribonuclease, such as RNase H. In other embodiments, cleavage steps may be carried out by treatment with a nickase.

“Cluster” as generally used herein means an amplicon or clonal population of a single polynucleotide amplified by a surface amplification technique, such as bridge PCR. In some embodiments, the term “cluster” includes amplicons produced by rolling circle amplification.

“Hydrogel” as generally used herein means a gel comprising a crosslinked hydrophilic polymer network with the ability to absorb and retain large amounts of water (for example, 60 to 90 percent water, or 70 to 80 percent) without dissolution due to the establishment of physical or chemical bonds between the polymeric chains, which may be covalent, ionic or hydrogen bonds. Hydrogels exhibit high permeability to the oxygen and nutrients, making them attractive materials for cell encapsulation and culturing applications. Hydrogels may comprise natural or synthetic polymers and may be reversible (i.e. degradable or depolymerizable) or irreversible. Synthetic hydrogel polymers can include polyethylene glycol (PEG), poly(2-hydroxyethyl methacrylate) and poly(vinyl alcohol). Natural hydrogel polymers can include alginate, hyaluronic acid and collagen. The following reference describe hydrogels and their biomedical uses: Drury et al, Biomaterials, 24:4337-4351 (2003); Garagorri et al, Acta Biomatter, 4 (5): 1139-1147 (2008); Caliari et al, Nature Methods, 13 (5): 405-414 (2016); Bowman et al, U.S. Pat. No. 9,631,092; Koh et al, Langmuir, 18 (7): 2459-2462 (2002).

A “tiling” as generally used herein means a set of sub-regions (referred to herein as “tiles”) that cover a surface without gaps or overlaps. As used herein, “without overlaps” means that the interiors of any two tiles are disjoint, and “without gaps” means that any sub-area of the surface is within the area of one or more tiles. In some embodiments, tile sub-regions are regular polygons. As used herein, “cover” comprises maximal coverings wherein a physical surface and regular polygons may have dimensions which do not permit an exact cover of a surface. For example, a covering may not include areas of a surface near its edges because of the size and shape of the surface and the regular polygons. In some embodiments, tiles comprise squares or hexagons. As used herein, a “quasi-tiling” is a set of tiles that covers a surface without overlaps. Thus, in a quasi-tiling there may be gaps between tiles. In some embodiments, the set of all quasi-tiling of a surface includes all tilings of the same surface, wherein the tiles are the same size and shape.

What is claimed is:

1. A method of making a spatially barcoded surface, comprising:
providing a surface comprising capture oligonucleotides attached thereto and a plurality of beads disposed thereon, wherein each bead comprises releasably attached barcode oligonucleotides each comprising a barcode sequence;
releasing said barcode oligonucleotides so that said barcode oligonucleotides are captured by said capture oligonucleotides; and
surface amplifying said captured barcode oligonucleotides to form clonal populations of said captured barcode oligonucleotides on said surface.

2. The method of claim 1, wherein said surface further comprises a diffusion inhibitor that occupies interstitial spaces between said beads.

3. The method of claim 1, wherein said plurality of said beads are closely spaced on said surface.

4. The method of claim 1, wherein said surface further comprises spacer beads.

5. The method of claim 4, wherein said spacer beads and said beads comprising said releasably attached barcode oligonucleotides are closely spaced on said surface.

6. The method of claim 1, wherein at least a portion of said barcode oligonucleotides have a 5′ phosphate, and wherein said method further comprises providing a splint oligonucleotide configured to form a duplex with a 5′-end and a 3′-end of said barcode oligonucleotides so that the 5′-end and the 3′-end are linked in the presence of a ligase to form a tandem barcode oligonucleotide whenever the 5′-end of said barcode oligonucleotide has a 5′ phosphate.

7. The method of claim 6, further comprising determining positions of said barcode oligonucleotides on said surface from said barcode sequences of said tandem barcode oligonucleotides.

8. The method of claim 6, further comprising determining positions of said barcode oligonucleotides on said surface from information derived from an image of said beads on said surface.

9. The method of claim 1, wherein said beads are hydrogel beads.

10. The method of claim 9, wherein said hydrogel beads are degradable.

11. The method of claim 1, wherein said barcode oligonucleotides are photo-releasable.

12. The method of claim 1, wherein said surface amplifying is carried out by bridge polymerase chain reactions.

13. A composition comprising a surface covered by a quasi-tiling, wherein each different tile of said surface has attached a different primary barcode oligonucleotide and at least one tandem barcode oligonucleotide from at least one adjacent tile.

14. The composition of claim 13, wherein said each different tile of said surface has attached said different primary barcode oligonucleotide and at least one tandem barcode oligonucleotide from each adjacent tile.

15. The composition of claim 13, wherein each of said tiles comprises a hexagon.

16. A surface divided into a plurality of sub-regions, wherein a sub-region of the plurality of sub-regions comprises:
(i) a first barcode oligonucleotide, wherein said first barcode oligonucleotide is spatially associated with said sub-region, and
(ii) at least one tandem barcode oligonucleotide, wherein said at least one tandem barcode oligonucleotide comprises at least a portion of said first barcode oligonucleotide and at least a portion of a second barcode oligonucleotide, wherein said second barcode oligonucleotide is spatially associated with an adjacent sub-region.

17. The surface of claim 16, wherein said plurality of sub-regions comprises a quasi-tiling of said surface.
